(19) United States
(12) Patent Application Publication
(10) Pub. No.: US 2003/0194705 A1
(43) Pub. Date: Oct. 16, 2003

(54) Methods of using unnatural nucleobases for decoding

(76) Inventor: Gary P. Schroth, Foster City, CA (US)

Correspondence Address: Pennie & Edmonds, LLP, 3300 Hillview Avenue, Palo Alto, CA, 94304, US

(21) Appl. No.: 10/087,411

(22) Filed: Mar. 1, 2002

(51) Int. Cl. C12Q 1/68; G06F 19/00; G01N 33/48; G01N 33/50

(52) U.S. Cl. 435/6; 702/20

(57) ABSTRACT

The present invention provides methods and compositions useful for coding and decoding complex mixtures of test units. These methods and compositions use coding and decoding oligonucleotides that comprise standard nucleobases and also non-standard nucleobases that selectively base pair with other non-standard nucleobases. These non-standard nucleobases nucleobases display little or no selective base pairing with standard nucleobases. The use of the non-standard nucleobases increases the diversity of the coding oligonucleotides and reduces the cross-reactivity of the coding and/or decoding oligonucleotides with other molecules.

### 1. FIELD OF THE INVENTION

The present invention relates to methods and compositions for coding and decoding a test unit in a plurality of test units.

### 2. BACKGROUND OF THE INVENTION

Modern biotechnology often demands high-throughput analysis of large numbers of samples. Randomly assembled arrays of nucleic acids and other molecules have been developed to facilitate such high-throughput analysis. Since molecules of randomly assembled arrays do not have to be assembled at specific sites, large numbers of molecules can be assembled into an array with minimal cost. The molecules of the array can then be assayed at one time for specific properties. However, in order for a randomly assembled array to be useful, the individual molecules of the array should be identifiable. This is typically accomplished by coding the array followed by a decoding process to identify molecules of the array. Improved compositions and methods for coding and decoding are needed to increase coding diversity and reduce nonspecific binding by coding molecules.

### 3. SUMMARY OF THE INVENTION

Embodiments of the present invention provide improved methods and compositions useful for coding and decoding complex mixtures of test units. A test unit can be coded by, for example, linking to the test unit or incorporating in the test unit a coding oligonucleotide, described below, that can be used to identify the test unit. Once coded, a test unit can be decoded by detecting the coding oligonucleotide thereby identifying the test unit.

These methods and compositions use coding and decoding oligonucleotides comprising an expanded “alphabet” of nucleobases, and, as a result, display increased diversity and/or reduced cross-reactivity with respect to mixtures coded with oligonucleotides made up of standard nucleobases (e.g. standard encoding nucleobases such as adenine, guanine, cytosine, thymine and uracil, and common analogs thereof). The expanded “alphabet” of nucleobases includes the standard nucleobases and also includes non-standard nucleobases that base pair with other non-standard nucleobases (“orthogonal nucleobases”). Significantly, the orthogonal nucleobases display little or no selective base pairing with standard nucleobases. The reduced or eliminated reactivity with standard nucleobases reduces the cross-reactivity of the coding and decoding oligonucleotides. For instance, in a coded mixture of test oligonucleotides that are to be probed for binding with target oligonucleotides, the coding and decoding oligonucleotides of the present invention display little or no cross-reactivity with the test oligonucleotides and target oligonucleotides.

In addition, coding and decoding oligonucleotides of the present invention can be significantly more diverse than oligonucleotides consisting of standard nucleobases. Oligonucleotides consisting of standard nucleobases are generally composed of an alphabet of only four nucleobases with unique base pairing properties, e.g. adenine, guanine, cytosine and either thymine or uracil, or common analogs thereof. In contrast, the coding and decoding oligonucleotides can comprise up to eight or more nucleobases with unique pairing properties. Such coding and decoding oligonucleotides can have greatly increased base pairing diversity when compared to similarly sized oligonucleotides of standard nucleobases. For example, a ten residue oligonucleotide composed of four nucleobases can have one of 4¹⁰ (approximately 10⁶) sequences with unique base pairing specificities, while a ten residue oligonucleotide composed of eight nucleobases can have one of 8¹⁰ (approximately 10⁹) sequences with unique base pairing specificities. Thus, increasing the “alphabet” of nucleobases from four to, for example, eight increases exponentially the information content of a given oligonucleotide. For the 10-mer example above, the information content increased by 10³. Coding oligonucleotides comprising an expanded alphabet of nucleobases can encode greater complexity than same-length oligonucleotides comprising only standard nucleobases (4-letter alphabet). As a consequence, to encode a given degree of complexity, the coding oligonucleotides of the invention can be significantly shorter than their standard counterparts.

In one aspect, embodiments of the present invention provide a method for identifying or isolating a coded test unit in a plurality of test units. In general, the test unit can be coded with a unique coding oligonucleotide comprising an orthogonal nucleobase. In certain embodiments, other test units of the plurality of test units can be coded with other unique coding oligonucleotides. A first test unit can comprise a first coding oligonucleotide, a second test unit can comprise a second coding oligonucleotide, and so on. The test unit can additionally comprise one or more test moieties. A test moiety can be any moiety known to those of skill in the art including, for instance, a small molecule, a peptide, a polypeptide, an oligonucleotide or a polynucleotide. Typically, a test unit can be used to assay one or more properties of the test moiety. Advantageously, test units that comprise the same test moiety can also comprise the same coding oligonucleotide so that all test units comprising the test moiety can be uniquely identified by the coding oligonucleotide.

The test units can comprise any material known to those of skill in the art to be capable of comprising coding oligonucleotides and/or test moieties. For instance, the test units can be molecules comprising coding oligonucleotides. In addition, the test units can be solid supports known to those of skill in the art. Such solid supports can comprise any material on which a coding oligonucleotide and/or a test moiety may be immobilized including porous substrates, metals, polymers, glasses, polysaccharides and the like. Supports may also take on any form including beads, disks, slabs, strips or any other form capable of bearing compounds. Coding oligonucleotides and/or test moieties can be immobilized to the substrate by any means known to one of skill in the art for immobilizing molecules.

According to embodiments of the method of the present invention, a test unit comprising a coding oligonucleotide can be decoded by contacting the test unit with a decoding oligonucleotide under conditions in which the decoding oligonucleotide and the coding oligonucleotide produce a detectable hybridization signal. The decoding oligonucleotide and the coding oligonucleotide can produce a detectable hybridization signal, by, for example, isolating the test unit from the remainder of the plurality of test units. They can also produce a detectable hybridization signal by any other means known to those of skill in the art. For instance, the signal can be a dye, a combination of dyes, a radioactive signal, an enzymatic signal, biotin or any other signal known to those of skill.

The decoding oligonucleotide typically complements the coding oligonucleotide such that the decoding oligonucleotide is capable of selectively hybridizing to the coding oligonucleotide under the decoding conditions. For instance, the decoding oligonucleotide can be perfectly complementary to a stretch of nucleotides of the coding oligonucleotide sufficient to generate a selective hybridization signal. Also for instance, the decoding oligonucleotide can comprise an orthogonal nucleobase complementary to, and at a position corresponding to, the orthogonal nucleobase of the coding oligonucleotide. If the coding oligonucleotide comprises a plurality of orthogonal nucleobases, then the decoding oligonucleotide can complement the coding oligonucleotide at positions corresponding to the orthogonal nucleobases of the coding oligonucleotide.

The decoding conditions will be apparent to those of skill in the art and can be chosen so that coding oligonucleotide of the test unit can selectively hybridize to the decoding oligonucleotide. Factors to be considered in choosing the decoding conditions include the length and degree of complementarity between the coding oligonucleotide and the decoding oligonucleotide, the G- and C- content of the oligonucleotides, the iso-G and iso-C content of the oligonucleotides and other factors that will be apparent to those of skill in the art.

In another aspect, embodiments of the invention provide a method for decoding coded test units. The method can advantageously be used to decode the test units of a randomly assembled, coded plurality of test units. For instance, a coded array of test units can be decoded with the method of the invention to determine the identity of test units of interest. A first coded test unit of the plurality of test units can be identified according to the above method. A second coded test unit of the plurality of test units can then be identified according to the above method. The method can then be repeated for each test unit to be decoded.

In another aspect, embodiments of the present invention provide kits for coding and/or decoding test units. The kits can comprise test units that can be used in the methods described above. Each test unit can comprise a coding oligonucleotide. Each test moiety can also comprise a test moiety or can be capable of being linked to a test moiety. The kits can also comprise a decoding oligonucleotide that corresponds to the coding oligonucleotide. In certain embodiments, the kits can comprise a plurality of test units or an array of test units.

The method and compositions of the present invention can be used to decode large, randomly assembled pluralities. A randomly assembled plurality of test units can thus be assayed for one or more desired properties en masse. Those test units that display the desired property or properties can then be identified or isolated by decoding the coding oligonucleotide of the test units. The use of orthogonal nucleobases both increases the diversity of the coding oligonucleotides and reduces the cross-reactivity of the coding and/or decoding oligonucleotides with other molecules. The methods and compositions of the present invention can be applied in any field that can benefit from screening randomly assembled pluralities including the fields of genotyping and gene expression profiling.

### 4. BRIEF DESCRIPTION OF THE FIGURES

FIG. 1A provides an example of a coded test unit;

FIG. 1B provides an example of a coded substrate comprising a test moiety and a coding oligonucleotide;

FIG. 1C provides an example of a coded substrate bearing a polynucleotide comprising a test oligonucleotide and a coding oligonucleotide; and

FIG. 2 provides standard nucleobases and several examples of orthogonal nucleobases of the present invention.

### 5. DETAILED DESCRIPTION OF THE PREFERRED EMBODIMENTS

As discussed in detail below, embodiments of the present invention provide novel methods and compositions for decoding pluralities of test units. The novel methods and compositions show significantly reduced cross-reactivity and significantly improved sequence diversity in their coding and/or decoding molecules. According to the methods and compositions described below, the coding and/or decoding molecules comprise an expanded alphabet of naturally occurring and synthetic nucleobases with unique base pairing properties to increase sequence diversity and to reduce cross-reactivity.

5.1 Abbreviations

The abbreviations used throughout the specification to refer to polynucleotides comprising specific nucleobase sequences are the conventional one-letter abbreviations. Thus, when included in a polynucleotide, the naturally occurring encoding nucleobases are abbreviated as follows: adenine (A), guanine (G), cytosine (C), thymine (T) and uracil (U). Certain non-standard nucleobases of the present invention, discussed in detail below, when included in a polynucleotide are abbreviated as follows: iso-guanine (iso-G), iso-cytosine (iso-C), 2,6-diaminopyrimidine (K) and xanthine (X). Also, unless specified otherwise, polynucleotide sequences that are represented as a series of one-letter abbreviations are presented in the 5′->3′ direction.

5.2 Definitions

As used herein, the following terms shall have the following meanings:

“Polynucleotide” and “oligonucleotide” are used interchangeably to refer to a polymer of natural or synthetic nucleobases, or a combination of both. Synthetic nucleobases specifically include the orthogonal nucleobases described in detail below. Other common synthetic nucleobases of which polynucleotides may be composed include 3-methlyuracil, 5,6-dihydrouracil, 4-thiouracil, 5-bromouracil, 5-thorouracil, 5-iodouracil, 6-dimethyl-aminopurine, 6-methyl aminopurine, 2-aminopurine, 2,6-diaminopurine, 6-amino-8-bromo purine, inosine, 5-methylcytosine, 7-deazaadenine, and 7-deazaguanosine. Additional non-limiting examples of synthetic nucleobases of which the target nucleic acid may be composed can be found in Fasman, CRC PRACTICAL HANDBOOK OF BIOCHEMISTRY AND MOLECULAR BIOLOGY, 1985, pp. 385-392; Beilstein's Handbuch der Organischen Chemie, Springer Verlag, Berlin and Chemical Abstracts, all of which provide references to publications describing the structures, properties and preparation of such nucleobases.

The backbone of a polynucleotide can be composed entirely of “native” phosphodiester linkages, or it may contain one or modified linkages, such as one or more phosphorothioate, phosphorodithioate, phosphoramidate or other modified linkages. As a specific example, a polynucleotide may be a peptide nucleic acid (PNA), which contains amide interlinkages. Additional examples of modified bases and backbones that can be used in conjunction with the invention, as well as methods for their synthesis can be found, for example, in U.S. Pat. No. 5,432,272; U.S. Pat. No. 6,001,983; Uhlman & Peyman, 1990, Chemical Review 90(4):544-584; Goodchild, 1990, Bioconjugate Chem. 1(3):165-186; Egholm et al., 1992, J. Am. Chem. Soc. 114:1895-1897; Gryaznov et al., J. Am. Chem. Soc. 116:3143-3144, as well as the references cited in all of the above.

“Standard nucleobases” refers to the encoding nucleobases found in naturally occurring polynucleotides known to those of skill in the art and includes the nucleobases A, G, C, T and U, and common analogs or derivatives thereof that are capable of forming selective base pairs with the encoding nucleobases.

“Non-standard nucleobases” refers to nucleobases other than the standard nucleobases. Typically, non-standard nucleobases can be incorporated into polynucleotides and are capable of forming base pairs with other nucleobases.

“Orthogonal nucleobases” refers to non-standard nucleobases that selectively form base pairs with other non-standard nucleobases in preference to forming base pairs with standard nucleobases. For instance, orthogonal nucleobases include nucleobases that have unique hydrogen bonding patterns relative to those of standard nucleobases. When incorporated into a single stranded polynucleotide, an orthogonal nucleobase is capable of forming a selective base pair with another orthogonal nucleobase. In particular, a single stranded polynucleotide comprising a first orthogonal nucleobase is capable of selectively hybridizing to a polynucleotide of complementary nucleobase sequence, including a complementary orthogonal nucleobase at a position corresponding to the first orthogonal nucleobase, under the appropriate conditions. In certain embodiments, the first polynucleotide is capable of hybridizing to the polynucleotide of complementary sequence under conditions known to those of skill in the art to discriminate between a perfect hybrid and a one base mismatch. Orthogonal nucleobases specifically include iso-C, iso-G, X, K and other orthogonal nucleobases described in U.S. Pat. No. 5,432,272, U.S. Pat. No. 5,965,364 and U.S. Pat. No. 6,001,983, the contents of which are hereby incorporated by reference.

“Coding” refers to a method of incorporating a coding oligonucleotide in a test unit or to a method of linking a coding oligonucleotide to a test unit.

“Decoding” refers to a method of identifying a test unit by identifying its coding oligonucleotide.

“Code oligonucleotide” or “coding oligonucleotide” refers to an oligonucleotide that can be used to identify a test unit. For example, a plurality of test units of ‘n’ unique members can be coded with ‘n’ unique coding oligonucleotides to identify each unique member of the plurality of test units.

“Decoding oligonucleotide” refers to an oligonucleotide that can be used to decode a test unit. Typically, a test unit is uniquely coded with a coding oligonucleotide. Hybridization of a decoding oligonucleotide to a corresponding coding oligonucleotide identifies the test unit. A decoding oligonucleotide corresponds to a coding oligonucleotide typically if the decoding oligonucleotide is capable of hybridizing to the coding oligonucleotide under decoding conditions. In certain embodiments a decoding oligonucleotide is complementary to a corresponding coding oligonucleotide.

“Substrate” refers to any solid support capable of having a code oligonucleotide and/or a test moiety immobilized thereon.

“Test moiety” refers to a moiety that can be assayed for a desired property. A test moiety can be assayed for a physical property, a chemical property or any other property known to those of skill in the art. For example, a test moiety can be assayed for an interaction with a target moiety, defined below. The identity of the test moiety is not critical for the invention. For instance, a test moiety can be an oligonucleotide that is to be assayed for binding to a second moiety. Other examples of test moieties include polypeptides, enzymes, substrates, receptors, ligands, nucleic acid binding proteins, carbohydrates and any other moiety having a measurable property known to those of skill in the art.

For convenience, in embodiments of the invention where two moieties are assayed, a first moiety can be referred to as the test moiety and a second moiety can be referred to as the target moiety. In particular, in embodiments of the invention where an immobilized moiety is assayed for an interaction with a moiety that is not immobilized, the immobilized moiety is generally referred to as the test moiety, and the moiety that is not immobilized is generally referred to as the target moiety, defined below. However, in certain embodiments of the invention the test moiety and/or the target moiety can be immobilized or not immobilized.

“Test unit” refers to any unit that can comprise a test moiety without limitation.

“Target molecule” or “target moiety” refers to a moiety that can be assayed for a desired property in the presence of a test moiety. The desired property can be a physical property, a chemical property or any other property known to those of skill in the art. The identity of the target moiety is not critical for the invention. For instance, a target moiety can be an oligonucleotide that is to be assayed for binding to a test moiety. Other examples to target moieties include polypeptides, enzymes, substrates, receptors, ligands, nucleic acid binding proteins carbohydrates and any other moiety known to those of skill in the art to have a measurable property.

“Coded test unit” refers to a test unit comprising a coding oligonucleotide or a test unit linked to a coding oligonucleotide.

“Coded substrate” refers to a substrate comprising a coding oligonucleotide or a substrate linked to a coding oligonucleotide.

5.3 Method of Identifying a Coded Test Unit

In one aspect, embodiments of the present invention provide a method that permits the selective identification of coded test units. According to the method, a coded test unit is contacted with a decoding oligonucleotide under conditions in which the decoding oligonucleotide produces a detectable hybridization signal. The coded test unit is coded with a coding oligonucleotide comprising an orthogonal nucleobase. The decoding oligonucleotide comprises an orthogonal nucleobase and has a sequence sufficiently complementary to the coding oligonucleotide to identify the coded test unit. Coded test units, coding oligonucleotides and decoding oligonucleotides are discussed in detail below.

5.3.1 The Coded Test Unit

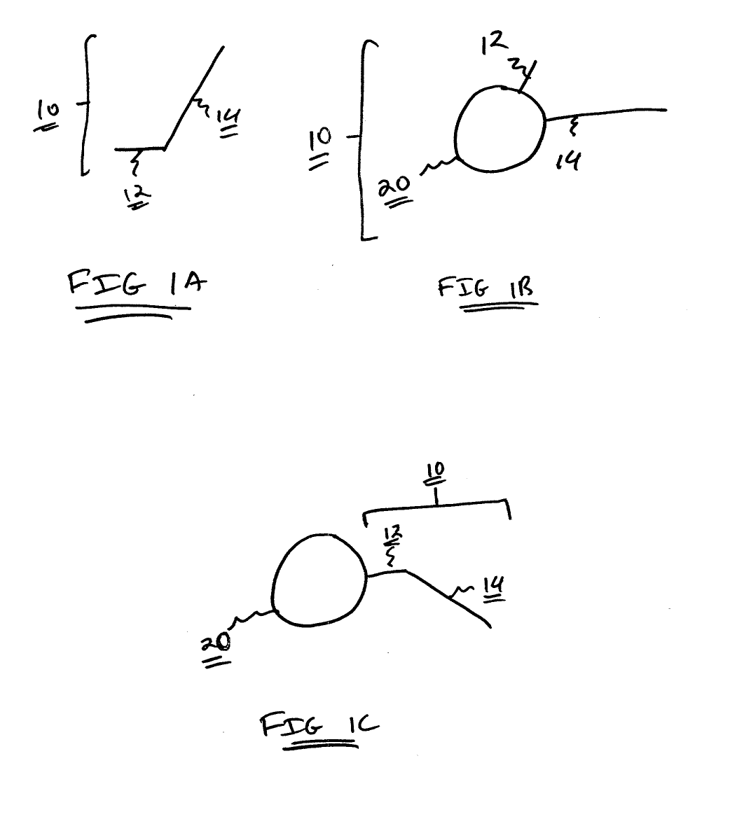

The methods of the present invention are useful for the identification of coded test units. Examples of coded test units are shown in FIG. 1A, FIG. 1B and FIG. 1C. In general, a coded test unit comprises a coding oligonucleotide and a test moiety.

Referring to FIG. 1A, coded test unit 10 comprises coding oligonucleotide 12 and test moiety 14. Coding oligonucleotide 12 is described in detail below. The identity of test moiety 14 is not critical. Test moiety 14 can be any moiety known to those of skill in the art including, for example, a small molecule, a macromolecule, a polymer, a polypeptide, an oligonucleotide or any other molecule that can be coded with coding oligonucleotide 12.

Coding oligonucleotide 12 can be linked to test moiety 14 by any means known to those of skill in the art. Coding oligonucleotide 12 can be linked by covalent linkage, by non-covalent association, by adsorption, or by any other technique known to those of skill. The linkage between coding oligonucleotide 12 and test moiety 14 can also be mediated by specific pairs of binding molecules such as biotin and streptavidin. The linkage between coding oligonucleotide 12 and test moiety 14 should not interfere with the coding function of coding oligonucleotide 12 and the function of test moiety 14.

In certain embodiments, coded test unit 10 can advantageously comprise a solid substrate. FIG. 1B presents an embodiment of coded test unit 10 wherein the link between test moiety 14 and coding oligonucleotide 12 is mediated by substrate 20. Coding oligonucleotide 12 is associated with substrate 20, and test moiety 14 is also associated with substrate 20. Coding oligonucleotide 12 and test moiety 14 can be independently associated with substrate 20 by any technique known to those of skill in the art for associating molecules on substrates. For example, coding oligonucleotide 12 and/or test moiety 14 can be adsorbed or otherwise non-covalently associated with substrate 20. Coding oligonucleotide 12 and/or test moiety 14 can also be covalently attached to substrate 20, or coding oligonucleotide 12 and/or test moiety 14 can be associated with substrate 20 through the mediation of specific binding pairs of molecules such as biotin and streptavidin. Covalent attachment of coding oligonucleotide 12 and test moiety 14 to substrate 20 is typical.

Substrate 20 can be any solid support to which compounds can be immobilized. The only requirement of substrate 20 is that coding oligonucleotides immobilized thereon be capable of selective hybridization with decoding oligonucleotides. Thus, substrate 20 can be a filter or a membrane, such as a nitrocellulose or nylon, glass, polymers such as polyacrylamide, gels such as agarose, dextran, cellulose, polystyrene, latex, or any other material known to those of skill in the art to which compounds can be immobilized. Advantageously, substrate 20 can be composed of a porous material such as those described in copending U.S. application Ser. No. 09/204,865 which is hereby incorporated by reference in its entirety. Exemplary porous materials include, for example, acrylic, styrene-methyl methacrylate copolymers, ethylene/acrylic acid and other porous materials described in detail in Ser. No. 09/204,865.

Substrate 20 can take on any form so long as the form does not prevent derivatization with compounds and does not prevent hybridization of coding oligonucleotides with decoding oligonucleotides. For instance, substrate 20 can have the form of disks, slabs, strips, beads, submicron particles, coated magnetic beads, gel pads, microtiter wells, slides, membranes, frits or other forms known to those of skill in the art. Substrate 20 is optionally disposed within a housing, such as a chromatography column, spin column, syringe-barrel, pipette, pipette tip, 96 or 384-well plate, microchannels, capillaries, etc., which aids the flow of liquids through the substrate. Additionally, materials having suitable average pore sizes and porosities are available commercially, and are either available in suitable thicknesses or can be cut into slabs, strips, disks or other convenient shapes of suitable thickness. In an embodiment of the invention, substrate 20 is an encoded microsphere of a plurality of microspheres such as those described in U.S. Pat. No. 6,023,540.

FIG. 1C presents an embodiment of a coded test unit associated with a solid substrate. In FIG. 1C, coded test unit 10 comprises coding oligonucleotide 12 and test moiety 14. Coded test unit 10 is associated with substrate 20. Coded test unit 10 can be associated with substrate 20 by any of the means for associating test moieties and/or coding moieties with a substrate 20 discussed above.

5.3.2 Coding Oligonucleotides and Decoding Oligonucleotides

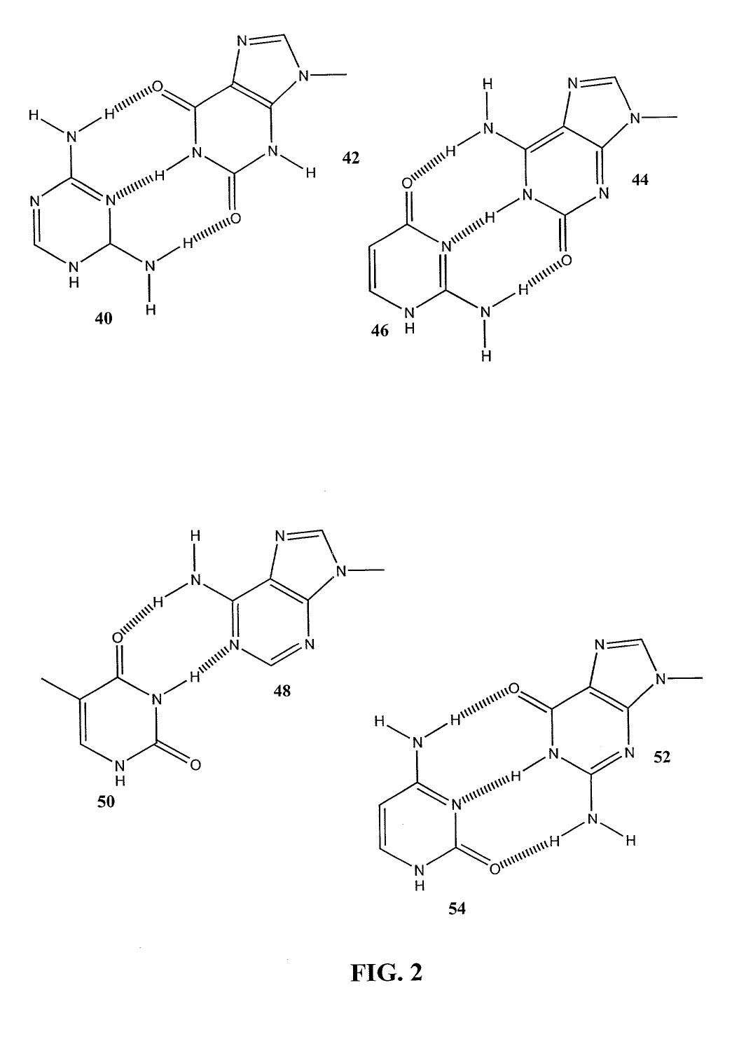

Coding oligonucleotide 12 is an oligonucleotide comprising an orthogonal nucleobase. Orthogonal nucleobases are non-standard nucleobases that are capable of selectively base pairing with other non-standard nucleobases. In certain embodiments, orthogonal nucleobases display little or no selective base pairing with standard nucleobases such as adenine, guanine, cytosine, thymine and uracil. Typical orthogonal nucleobases are illustrated in FIG. 2 and are discussed in detail in U.S. Pat. No. 5,432,272, U.S. Pat. No. 5,965,364 and U.S. Pat. No. 6,001,983, the contents of which are hereby incorporated by reference.

FIG. 2 illustrates four exemplary orthogonal nucleobases of the present invention and four standard nucleobases. While not intending to be bound by any particular theory, it is believed that an orthogonal nucleobase selectively base pairs with its complementary orthogonal nucleobase because of their unique complementary patterns of hydrogen bond donors and acceptors. To illustrate, standard nucleobase adenine 48 forms a selective base pair with standard nucleobase thymine 50 via two hydrogen bonds. Standard nucleobase adenine 48 has one hydrogen bond donor and one hydrogen bond acceptor (donor-acceptor) that complements a hydrogen bond acceptor and a hydrogen bond donor (acceptor-donor) of standard nucleobase thymine 50. Similarly, standard nucleobase guanine 52 has one hydrogen bond acceptor and two hydrogen bond donors (acceptor-donor-donor) that complement one hydrogen bond donor and two hydrogen bond acceptors (donor-acceptor-acceptor) of standard nucleobase cytosine 54. Orthogonal nucleobase xanthine 42 has a hydrogen bonding pattern distinct from the hydrogen bonding patterns of standard nucleobase adenine 48 and standard nucleobase guanine 52, and complementary orthogonal nucleobase 2,6-diaminopyrmidine 40 has a hydrogen bonding pattern distinct from those of standard nucleobase thymine 50 and standard nucleobase cytosine 54. The hydrogen bonding pattern of xanthine 42, acceptor-donor-acceptor, complements the hydrogen bonding pattern of 2,6-diaminopyrmidine 40, donor-acceptor-donor. Orthogonal nucleobase iso-guanine 44 has a hydrogen bonding pattern, donor-donor-acceptor, that complements the hydrogen bonding pattern of iso-cytosine 46, acceptor-acceptor-donor. The hydrogen bonding patterns of iso-guanine 44 and iso-cytosine 46 are distinct from those of the standard nucleobases.

Those of skill in the art will recognize that xanthine 42, 2,6-diaminopyrmidine 40, iso-guanine 44 and iso-cytosine 46 are four examples of the orthogonal nucleobases of the present invention. Orthogonal nucleobases include any nucleobase that can be incorporated into a polynucleotide and that displays selective base pairing for another orthogonal nucleobase relative to the standard nucleobases. Orthogonal nucleobases include, for instance, derivatives of xanthine 42, 2,6-diaminopyrmidine 40, iso-guanine 44 and isocytosine 46, analogs of xanthine 42, 2,6-diaminopyrmidine 40, iso-guanine 44 and isocytosine 46, and other orthogonal nucleobases such as H, J, M and N described in U.S. Pat. No. 5,432,272. Orthogonal nucleobases also include any other nucleobase that is capable of selective base pairing with one or more other orthogonal nucleobases.

Orthogonal nucleobases can be prepared by synthetic techniques known to those of skill in the art including, for instance, those described in U.S. Pat. No. 5,423,272, U.S. Pat. No. 5,965,364 and U.S. Pat. No. 6,001,983. Coding oligonucleotides can be prepared according to any method known to those of skill in the art for preparing oligonucleotides comprising non-standard nucleobases. For instance, such oligonucleotides can be prepared enzymatically or synthetically by standard techniques known to those of skill in the art including, for instance, solid phase techniques

A decoding oligonucleotide is an oligonucleotide comprising an orthogonal nucleobase that can be used to identify a coded test unit. Typically, a decoding oligonucleotide is sufficiently complementary to a corresponding coding oligonucleotide such that the decoding oligonucleotide is capable of selectively hybridizing to the coding oligonucleotide. The decoding oligonucleotide can comprise an orthogonal nucleobase complementary to, and at a position corresponding to, an orthogonal nucleobase of the corresponding coding oligonucleotide. In certain embodiments, the decoding oligonucleotide is perfectly complementary to a stretch of oligonucleotide in the coding oligonucleotide. The decoding oligonucleotide can complement, for example, a stretch of 6, 8, 10, 12, 15 or 20 or more nucleobases of the coding oligonucleotide. In certain embodiments, the decoding oligonucleotide can complement a stretch of 12-20 nucleobases of the coding oligonucleotide. The orthogonal nucleobases of the decoding oligonucleotide can be prepared by the techniques discussed above. The decoding oligonucleotide can also be prepared by techniques discussed above.

5.3.3 Kits for Decoding a Plurality of Test Units

Embodiments of the present invention provide kits for decoding a plurality of test units. The kits typically comprise a coded test unit, such as a coded substrate, and one or more decoding oligonucleotides. The coded substrate typically comprises a coding oligonucleotide according to the description above. The decoding oligonucleotide typically corresponds to the coding oligonucleotide according to the description above. The decoding oligonucleotide can be used to decode a test unit linked to the coded substrate. In certain embodiments, the kit comprises coded substrate and a plurality of decoding oligonucleotides wherein the coded substrate comprises a plurality of coding oligonucleotides corresponding to the decoding oligonucleotides.

5.3.4 Contacting Coded Test Unit with Decoding Oligonucleotide

According to the method, the coded test unit of the plurality of test units is contacted with the decoding oligonucleotide under conditions in which the decoding oligonucleotide generates a hybridization signal sufficient to distinguish the coded test unit from other test units of the plurality of test units. The coded test unit comprises a coding oligonucleotide that sufficiently complements the decoding oligonucleotide to selectively identify the coded test unit among the rest of the plurality of test units, as discussed above.

The conditions under which the coded test unit of the plurality of test units is contacted with the decoding oligonucleotide depend upon the sequence of the coding oligonucleotide and the sequence of the decoding oligonucleotide and will be apparent to one of skill in the art. For instance, the extent and degree of sequence complementary, and the G/C/iso-G/iso-C content of the complementary regions of the oligonucleotides will influence the ideal contact conditions. The contact conditions should be conditions under which the coding oligonucleotide and the decoding oligonucleotide selectively hybridize to form a complex. Specific conditions for capture including polynucleotide concentration, volumes, pH, buffer, salt concentration, incubation time, temperature and so forth are within the knowledge of those of skill in the art. Typically, a DNA coding oligonucleotide can be contacted with a DNA decoding oligonucleotide in, for example, 100 mM NaCl or 100 mM ammonium acetate at a pH of, for example, about 6 to about 8. Much lower salt concentrations can be used for PNA-PNA, PNA-RNA or PNA-DNA pairs. If the pair is PNA-PNA, very little or no salt can be used in the capture conditions.

As the decoding oligonucleotide contacts the plurality of test units, selective binding between the decoding oligonucleotide and a sufficiently complimentary coding oligonucleotide of the plurality of test units takes place. Thus, the decoding oligonucleotide can contact the plurality of test units for a period of time that is long enough for binding to occur. The kinetics of binding will depend on many factors. For instance, the factors can include the GC or iso-G/iso-C content the decoding oligonucleotide, the lengths of the decoding oligonucleotide and coding oligonucleotide, the amount of the test unit, the of the decoding oligonucleotide, the salt and/or buffer conditions of the sample, the temperature of hybridization, etc. Such conditions will be apparent to one of skill in the art.

The test unit can be identified by the detection of a detectable hybridization signal from the decoding oligonucleotide. For instance, in an embodiment of the invention, a coded test unit can be identified by isolating the coded test unit from a plurality of molecules. The coded test unit can be contacted with a decoding molecule that is, for instance, immobilized on a solid substrate under conditions in which the coded test unit hybridizes to the decoding oligonucleotide. The remainder of the plurality of test units can be removed and the decoding oligonucleotide can optionally be washed to remove any non-selectively bound molecules. The coded test unit can then be detected and/or used by any technique known to those of skill in the art. Other techniques for isolating a coded test unit by hybridization to a decoding oligonucleotide will be apparent to those of skill in the art.

The test unit can also be identified by detection of other hybridization signals known to those of skill in the art. For instance, the decoding oligonucleotide and/or the coding oligonucleotide can be labeled with a detectable label known to those of skill in the art. Such labels include dyes, radioactive labels, members of specific binding pairs such as biotin and avidin and other labels known to those of skill in the art. After the decoding oligonucleotide and/or the coded test unit is washed to remove non-selectively bound molecules, the label can be detected to identify the hybridized oligonucleotides and thereby the coded test unit.

A plurality of test units can be decoded according to the method of the present invention. The plurality of test units can be any plurality of test units that is coded by coding oligonucleotides. A first test unit can be identified by the method of the present invention as described above. A second test unit can then be identified from the remainder of the plurality of test units according to the methods of the present invention thereby decoding a first and a second test unit. A plurality of test units of any size can be decoded by the methods of the present invention. The coding and decoding oligonucleotides should of sizes sufficient to uniquely identify each unique test unit. For instance, by using an alphabet of eight nucleobases, an coding oligonucleotides with a length of ten or more nucleobases can be used to uniquely identify 10⁹ unique test units. Those of skill in the art can readily determine the size of coding and decoding oligonucleotides necessary to code and decode a plurality of test units of a given size.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below.

All references cited herein are hereby incorporated by reference in their entirety.

1. A method of identifying a coded test unit in a plurality of coded test units comprising the step of:
contacting the coded test unit with a decoding oligonucleotide comprising an orthogonal nucleobase under conditions in which the decoding oligonucleotide produces a detectable hybridization signal sufficient to distinguish the coded test unit from the remainder of the plurality of coded test units.

2. A method for decoding a plurality of coded test units comprising the steps of:
a. identifying a first molecule in the plurality of coded test units according to the method of claim 1; and
b. identifying a second substrate in the plurality of coded test units according to the method of claim 1.

3. The method of claim 1 wherein the coded test unit is coded with a decoding oligonucleotide comprising an orthogonal nucleobase.

4. The method of claim 1 wherein the plurality of coded test units are coded with decoding oligonucleotides, wherein each decoding oligonucleotide independently comprises an orthogonal nucleobase.

5. The method of claim 1, 2, 3 or 4 wherein the orthogonal nucleobase is iso-C, iso-G, K, X or H.

6. The method of claim 1 wherein the coded test unit comprises a solid substrate.

7. A method for decoding a plurality of coded substrates comprising the steps of:
a. identifying a first substrate in the plurality of coded substrates according to the method of claim 6; and
b. identifying a second substrate in the plurality of coded substrates according to the method of claim 6.

8. The method of claim 6 wherein each coded substrate comprises a test moiety.

9. The method of claim 8 wherein the test moiety is an oligonucleotide.

10. The method of claim 9 wherein a single polynucleotide comprises the test moiety and the coding oligonucleotide.

11. The method of claim 9 wherein a first polynucleotide comprises the test moiety and a second polynucleotide comprises the coding oligonucleotide.

12. The method of claim 6 wherein the plurality of coded substrates is in an array.

13. A coded substrate comprising a test moiety and a coding oligonucleotide, said coding oligonucleotide comprising an orthogonal nucleobase.

14. The coded substrate of claim 13 wherein the orthogonal nucleobase is iso-C, iso-G, K, X or H

15. The coded substrate of claim 13 wherein the test moiety is an oligonucleotide.

16. The coded substrate of claim 15 wherein a polynucleotide comprises the test moiety and the coding oligonucleotide.

17. The coded substrate of claim 15 wherein a first polynucleotide comprises the test moiety and a second polynucleotide comprises the coding oligonucleotide.

18. A plurality of coded substrates according to claim 13.

19. An array of coded substrates according to claim 13.

20. A kit for decoding a plurality of test units comprising a coded substrate according to claim 13 and a decoding oligonucleotide.
